(19) United States
(12) Patent Application Publication
(10) Pub. No.: US 2022/0227797 A1
(43) Pub. Date: Jul. 21, 2022

(54) TUNGSTEN IMIDO ALKYLIDENE O-BITET AND O-BINOL COMPLEXES AND USE THEREOF IN OLEFIN METATHESIS REACTIONS

(71) Applicant: Verbio Vereinigte Bioenergie AG, Zörbig (DE)

(72) Inventors: Levente Ondi, Budapest (HU); Csaba Hegedüs, Budapest (HU); Ágota Bucsai, Budapest (HU); Jeno Varga, Budapest (HU); Benedek Vakulya, Budapest (HU); Krisztian Lorincz, Budapest (HU); Henrik Gulyas, Budapest (HU); Hasan Mehdi, Budapest (HU)

(21) Appl. No.: 17/595,761

(22) PCT Filed: May 27, 2020
(86) PCT No.: PCT/EP2020/064743

§ 371 Date: Nov. 23, 2021

(30) Foreign Application Priority Data
May 27, 2019 (EP) 19176783.9

(51) Int. Cl. C07F 11/00; B01J 31/22; B01J 31/18; C07C 67/475; C07D 211/02

(52) U.S. Cl. CPC C07F 11/00; B01J 31/2265; B01J 31/181; B01J 2231/54; C07C 67/475; C07D 211/02; B01J 2531/66; B01J 31/2208

(57) ABSTRACT

The invention relates to tungsten imido alkylidene compounds bearing a ligand derived from a 1,1′-binaphthyl-2-ol or a 5,5′,6,6′,7,7′,8,8′-octahydro-1,1′-binaphthyl-2-ol which bind to tungsten in its olate-form via proton abstraction from the phenolic OH group. The complexes may be used in various olefinic metathesis reactions, preferably ethenolysis and cross-metathesis of unsaturated fatty acid esters, and ring-closing metathesis reactions.

### FIELD OF THE INVENTION

The invention relates to tungsten imido alkylidene O-bitet complexes, wherein the term “O-bitet” as used within this disclosure means a ligand derived from 5,5′,6,6′,7,7′,8,8′-octahydro-1,1′-binaphthyl-2-ol which binds to tungsten in its olate-form via proton abstraction from the phenolic OH group. In another embodiment, the bitet ligand is used in its aromatic form, i.e. it is derived from a 1,1′-binaphthyl-2-ol, herein termed as “O-binol”. The complexes may be used in various olefin metathesis reactions, preferably in ethenolysis and cross-metathesis such as cross-metathesis of unsaturated fatty acid esters, and in ring-dosing metathesis reactions.

### BACKGROUND OF THE INVENTION

Olefin metathesis reactions catalyzed by transition metal catalysts are among the most important reactions of organic synthetic chemistry. A valuable type of known catalysts is the group of metal imido alkylidene complexes. The efficacy thereof is depending on the type of metal, alkylidene group and ligands. However, until now, knowledge of respective structure-activity relationships between such catalysts and substrate to be metathesized is limited. Consequently, the selection, synthesis and use of a catalyst in a particular metathesis reaction generally requires a research program in order to find the optimum.

### OBJECTS OF THE INVENTION

It is the object of the invention to provide a group of tailor-made and closely related metal imido alkylidene compounds or groups of closely related metal imido alkylidene compounds which are designed such to be efficient in olefin metathesis reactions, and preferably efficient in ethenolysis and cross-metathesis such as cross-metathesis of unsaturated fatty acid esters, and in ring-closing metathesis reactions.

### SUMMARY OF THE INVENTION

This object has been achieved with particular tungsten imido alkylidene O-bitet and O-binol complexes and methods using the complexes as defined in the appended independent claims. Preferred embodiments are specified in the claims dependent thereon.

The alkylidene moiety of he tungsten alkylidene complexes is designed either to be based on

═CH—C(CH₃)₂—C₆H₅ [herein denoted as compounds of formula (I)], or

═CH—C(CH₃)₂-phenyl wherein the phenyl ring bears (or comprises) in o-position a group selected from O—(C₁-C₆ alkyl) and —CH₂—O—(C₁-C₆ alkyl) [herein denoted as compounds of formula (II)], or

═CH-phenyl, wherein the phenyl ring bears (or comprises) in o-position a group selected from O—(C₁-C₆ alkyl) and —CH₂—O—(C₁-C₆ alkyl) [herein denoted as compounds of formula (III)], or

═C(phenyl)₂, wherein at least one of the phenyl rings bears (or comprises) in o-position a group selected from O—(C₁-C₆ alkyl) and —CH₂—O—(C₁-C₆ alkyl), respectively [herein denoted as compounds of formula (IV)], or

═CH—Ar, wherein Ar [herein denoted as compounds of formula (VI)] is selected from phenyl [herein denoted as compounds of formula VI-A], naphthyl [herein denoted as compounds of formula VI-B] and anthracenyl [herein denoted as compounds of formula VI-C]. Preferably, when Ar=phenyl, i.e. the tungsten alkylidene moiety is ═CH—C₆H₅, the phenyl residue is unsubstituted or may be substituted but does not bear (or does not comprise) in o-position a O—(C₁-C₆ alkyl) group.

The imido residue preferably is a phenyl imido residue.

Preferably, said phenyl imido residue is substituted with electron-withdrawing groups such as halogen or trifluoromethyl, e.g. the phenyl residue being 2,6-dichlorophenyl, pentafluorophenyl or o-trifluoromethylphenyl.

The inventors discovered that such compounds may provide for excellent activity in various olefin metathesis reactions such as ethenolysis and cross-metathesis of unsaturated fatty acid esters, and in ring-closing metathesis reactions.

Without being bound by theory, the inventors assume that the combination of selected metal, i.e. tungsten, phenyl-containing alkylidene moieties, O-bitet ligand or O-binol ligand and imido ligand provide for a beneficial structure-activity relationship between the catalysts and substrate to be metathesized.

### DETAILED DESCRIPTION OF THE INVENTION

### Compounds Comprising a ═CHC(CH₃)₂C₆CH₅ Moiety of formula (I)

According to a first aspect, the invention relates to a compound of formula (I)

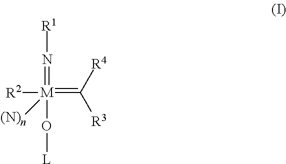

wherein

### M=W;

R¹ is selected from phenyl substituted with one or more of halogen or CF₃;
R²is selected from pyrrol-1-yl or indol-1-yl, optionally substituted, respectively;
one of R³ and R⁴ is H, and the other is C(CH₃)₂C₆H₅;

### LO— is

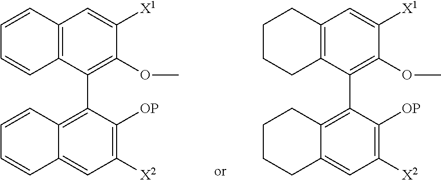

wherein X¹ and X² are independently selected from halogen, CF₃ and C₆F₆; or
X¹=X²=halogen, CF₃ or C₆F₅;
P is C₁-C₆ alkyl, or a silyl group; and
N is a neutral ligand bound to M,
wherein n is 1 or 2, when LO— is a O-bitet ligand, or
wherein n is 0, 1, or 2, when LO— is a O-binol ligand.

In a preferred embodiment, R¹ is 2,6-dichlorophenyl, pentafluorophenly, or o-CF₃-C₆H₄.

If not otherwise stated, the term “pyrrol-1-yl or indol-1-yl, optionally substituted” as used throughout this disclosure of all aspects defined herein, means that respective substituents may be selected from one or more of C₁₋₄ alkyl, C₁₋₄ alkoxy, halogen, nitrile, and phenyl.

In a preferred embodiment, R² is selected from the group consisting of pyrrol-1-yl, 2,5-dimethyl-pyrrol-1-yl, 2,5-diethyl-pyrrol-1-yl, 2,5-diphenyl-pyrrol-1-yl, and indol-1-yl.

In one embodiment, substituted indol-1-yl is 2-methyl-indol-1-yl.

It is known that LOH may exist in various optical forms, i.e. in racemic form and in the form of the enantiomers, i.e. in (R) and (S) form. The use of either the (R) or (S) enantiomer for forming the O-bitet ligand in the compound of formula (I) may be advantageous if the product resulting from the metathesis reaction is chiral. Then, the formation of an optically active form of the metathesis product may be possible, if desired.

If the formation of an optically active form is not desired, then the use of LOH in its racemic form for forming the bitet ligand in the compound of formula (I) is preferred. This is advantageous under economical aspects since racemic LOH typically is typically cheaper compared to its isolated enantiomers.

In one embodiment, LO— has (R) configuration.

In another embodiment, LO— has (S) configuration

In another embodiment, LO— is racemic.

The term “silyl” used in connection with P in the OP moiety may be any silyl group forming a covalent bond between silicon and oxygen.

Known groups are e.g. t-butyldimethylsilyl (TBS, TBDMS), trimethylsilyl (TMS), triethylsilyl (TES), triisopropylsilyl (TIPS), t-butyldiphenylsilyl (TBDPS), and triphenylsily.

In a preferred embodiment, said neutral ligand N is a nitrile.

Preferably, said nitrile is acetonitrile.

Nitrile binds via N to M.

In another preferred embodiment, said neutral ligand N is a phosphine.

Preferably, said phosphine is selected from the group consisting of dimethylphenyl phosphine, methyldiphenyl phosphine and tris(cyclohexyl) phosphine.

Phosphine binds via P to M.

In a further preferred embodiment, said neutral ligand is a pyridine.

Preferably, said pyridine is pyridine as such, or 2,2′-bipyridine, or 1,10-phenanthroline.

Said pyridine may be substituted with one or more substituents independently selected from C₁₋₄ alkyl, C₁₋₄ alkoxy, phenyl, phenoxy and halogen.

Said pyridine binds via N to M, either as a monodentate ligand such as pyridine as such, or as a bidentate ligand such as 2,2′-bipyridine and 1,10-phenanthroline.

Exemplified compounds of formula (I) are e.g. O-bitet complexes 1, 2, 3 and 17, 18 and 19:

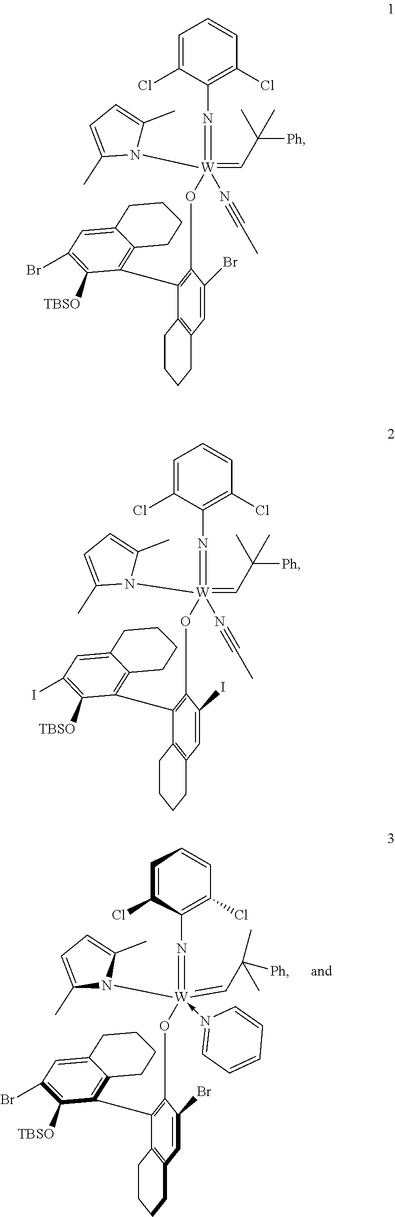

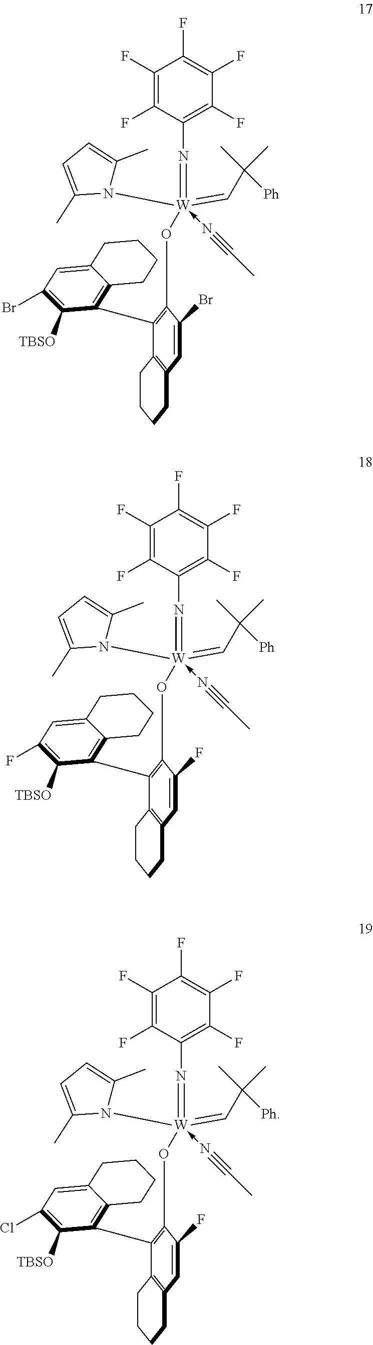

The compounds of formula (I) can be prepared from respective complexes not bearing a neutral ligand by subjecting same to said neutral ligand, respectively are made in presence of the ligand according to known methods.

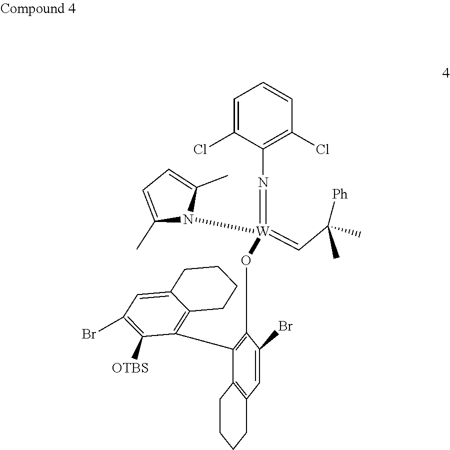

has been developed by XiMo AG/Hungary and is known from WO 2014/139679, where it is used for homo-metathesis of allyl benzene (compound 207 in Table 16 thereof). Herein, the bitet ligand LO— is provided as R-enantiomer.

The compound is further known from claim 26 of WO 2017/087710 (Provivi Inc). This reference discloses cross-metathesis between two internal olefins using compound 4 to produce pheromones.

Frequently, the complexes not bearing a neutral ligand such as compound 4 are present in non-crystallized form or in oily form after synthesis or even have to be prepared in situ when used in a metathesis reaction. Attempts to transfer oily forms into solid forms typically result in severe yield loss which is not acceptable under economic and industrial requirements.

However, complexed with a neutral ligand such as a nitrile such as acetonitrile, the complex may be provided in crystallized form. This is advantageous e.g. in view of the handling, efficacy of the compound in a metathesis reaction and commercial aspects.

Surprisingly, it has also been discovered that compound 4 provided with LO— as racemate crystallizes very well, contrary to the compound developed with R-LO.

In a further embodiment of the first aspect, an exemplified compound of formula (I) is O-binol compound 5:

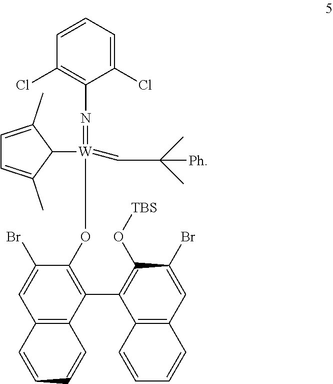

In a further aspect regarding the compound of formula (I), R³ may also be C₁₋₅ alkyl, wherein the other residues have the meaning as defined above with respect to said compound of formula (I).

### Compounds Comprising a ═CHC(CH₃)₂Phenyl Moiety wherein the Phenyl Residue Comprises in o-Position a Group Selected from O—(C₁₋₆ Alkyl) and —CH₂—O—(C₁₋₆ Alkyl)

According to a second aspect, the invention relates to a compound of formula (II)

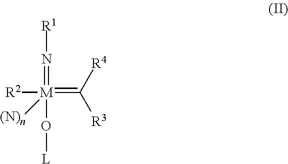

wherein

### M=W;

R¹ is selected from aryl, alkyl and cycloalkyl, each of which is optionally substituted;
R² is selected from pyrrol-1-yl and indol -1-yl, optionally substituted, respectively;
one of R³ and R⁴ is H, and the other is C(CH₃)₂phenyl, wherein the phenyl group of the C(CH₃)₂phenyl-moiety is additionally substituted in o-position with a group selected from O—(C₁-C₆ alkyl) and —CH₂—O—(C₁-C₆ alkyl);

### LO— is

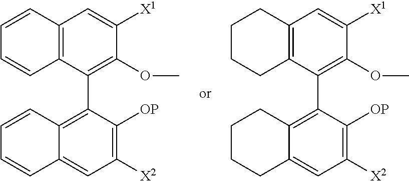

wherein X¹ and X² are independently selected from halogen, CF₃ and C₆F₆; or
X¹=X²=halogen, CF₃ or C₆F₅;
P is C₁-C₆ alkyl, or a silyl group; and
N is a neutral ligand bound to M, wherein n is 0, 1 or 2.

In a preferred embodiment, R¹ is selected from the group consisting of phenyl substituted with one or more of C₁-C₆ alkyl, O—(C₁-C₆ alkyl), phenyl, halogen and CF₃; t-butyl, and 1-adamantyl.

In one embodiment, R¹ is selected from phenyl substituted with one or more of halogen or CF₃.

In a preferred embodiment, R¹ is 2,6-dichlorophenyl, pentafluorophenly or o-CF₃-C₆H₄.

Preferably, R² is selected from pyrrol-1-yl, 2,5-dimethyl-pyrrol-1-yl, 2,5-diethyl-pyrrol-1-y,l2,5-diphenyl-pyrrol-1-yl, and indol-1-yl

In one embodiment, LO— has (R) configuration.

In another embodiment, LO— has (S) configuration

In another embodiment, LO— is racemic.

The use of racemic LO— may be advantageous under economical aspects since racemic LOH typically is typically cheaper compared to its enantiomers.

The term “silyl” used in connection with P in the OP moiety may be any silyl group forming a covalent bond between silicon and oxygen.

Known groups are e.g. t-butyldimethylsilyl (TBS, TBDMS), trimethylsilyl(TMS), triethylsilyl (TES), triisopropylsilyl (TIPS), t-butyldiphenylsilyl (TBDPS), and triphenylsily.

In a preferred embodiment, said neutral ligand N is a nitrile.

Preferably, said nitrile is acetonitrile.

Nitrile binds via N to M.

In another preferred embodiment, said neutral ligand N is a phosphine.

Preferably, said phosphine is selected from the group consisting of dimethylphenyl phosphine, methyldiphenyl phosphine and tris(cyclohexyl)phosphine.

Phosphine binds via P to M.

In a further preferred embodiment, said neutral ligand is a pyridine.

Preferably, said pyridine is pyridine as such, or 2,2′-bipyridine, or 1,10-phenanthroline.

Said pyridine may be substituted with one or more substituents independently selected from C₁₋₄ alkyl, C₁₋₄ alkoxy, phenyl, phenoxy and halogen.

Said pyridine binds via N to M, either as a monodentate ligand or a bidentate ligand.

### Compounds Comprising a ═CHphenyl Moiety wherein the Phenyl Residue Comprises in o-Position a Group Selected from O—(C₁₋₆ Alkyl) and —CH₂—O—(C₁₋₆ Alkyl)

According to a third aspect, the invention relates to a compound of formula (III)

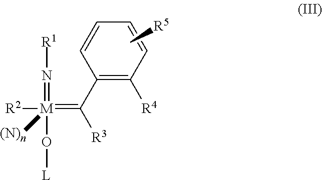

wherein

### M is W;

R¹ is selected foam aryl, alkyl and cycloalkyl, each of which is optionally substituted;
R² is pyrrol-1-yl or indol-1yl, optionally substituted, respectively;
R³ is selected from H;
R⁴ is selected from O—(C₁-C₆ alkyl), and —CH₂—O—(C₁-C₆ alkyl);
R⁵ is/are one or more residues independently selected from H, C₁-C₆ alkyl, O—(C₁-C₆ alkyl), phenyl, halogen, NO₂, CN, and NHC(O)—(C₁-C₆ alkyl);

### LO— is

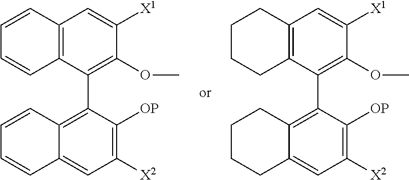

wherein
X¹ and X² are independently selected from halogen, CF₃ and C₆F₅; or
X¹=X²=halogen, CF₃or C₆F₅;
P is C₁-C₆ alkyl, or a silyl group; and
N is a neutral ligand bound to M, wherein n is 0, 1 or 2;
under the proviso that a compound of formula

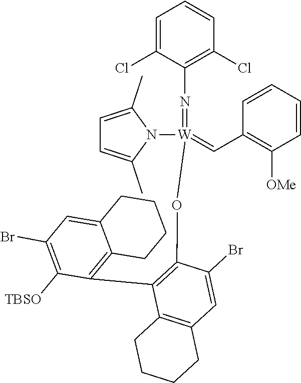

is excluded. The excluded compound (termed as compound 6) was developed by and is available from XiMo Ag/Hungary. Herein, the aryloxy residue LO— is in the R-form.

In a preferred embodiment, R¹ is selected from the group consisting of phenyl substituted with one or more of C₁-C₆ alkyl, O—(C₁-C₆ alkyl), phenyl, halogen and CF₃; t-butyl, and 1-adamantyl.

In one embodiment, R¹ is selected from phenyl substituted with one or more of halogen or CF₃.

In a preferred embodiment, R¹ is 2,6-dichlorophenyl, pentafluorophenly or o-CF₃-C₆H₄.

Preferably, R² is selected from pyrrol-1-yl, 2,5-dimethyl-pyrrol-1-yl, 2,5-diethyl-pyrrol-1-yl, 2,5-diphenyl-pyrrol-1-yl, and indol-1-yl.

In one embodiment, LO— has (R) configuration.

In another embodiment, LO— has (S) configuration

In another embodiment, LO— is racemic.

The use of racemic LO— may be advantageous under economical aspects since racemic LOH typically is typically cheaper compared to its enantiomers.

The term “silyl” used in connection with P in the OP moiety may be any silyl group forming a covalent bond between silicon and oxygen.

Known groups are e.g. t-butyldimethylsilyl (TBS, TBDMS), trimethylsilyl (TMS), triethylsilyl (TES), triisopropylsilyl (TIPS), t-butyldiphenylsilyl (TBDPS), and triphenylsilyl.

In a preferred embodiment, said neutral ligand N is a nitrile.

Preferably, said nitrile is acetonitrile.

Nitrile binds via N to M.

In another preferred embodiment, said neutral ligand N is a phosphine.

Preferably, said phosphine is selected from the group consisting of dimethylphenyl phosphine, methyldiphenyl phosphine and tris(cyclohexyl) phosphine.

Phosphine binds via P to M.

In a further preferred embodiment, said neutral ligand is a pyridine.

Preferabyl, said pyridine is pyridine as such, or 2,2′-bipyridine, or 1,10-phenanthroline.

Said pyridine may be substituted with one or more substituents independently selected from C₁₋₄ alkyl, C₁₋₄ alkoxy, phenyl, phenoxy and halogen.

Said pyridine binds via N to M, either as a monodentate ligand or a bidentate ligand.

The disclaimed compound (herein termed as compound 6) bearing a methoxy-substituted phenyl carbene is e.g. known from claim 27 of WO 2017/087710 (Provivi Inc). This reference discloses cross-metathesis between two internal olefins using the disclaimed compound to produce pheromones.

The new compounds of structure (III) can be made according to known methods, e.g. via alkylidene exchange as disclosed in WO 2015/155593 (XiMo AG). Prior to the carbene exchange, the O-bitet ligand may be introduced into the complex by reacting a bispyrrolide with e.g. a lithium salt LOLi according to known methods.

In a preferred embodiment, the compound of formula (III) is selected from a compound, wherein

M=W, R¹=2,6-dichlorophenyl; R²=2,5-dimethyl-pyrrol-1-yl; R³=H; R⁴=OCH₃; R⁵=H; X¹=X²=F; P=TBS (compound 7):

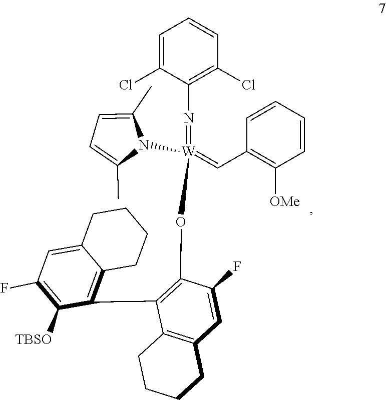

M=W, R¹=2,6-dichlorophenyl; R²=2,5-dimethyl-pyrrol-1-yl; R³=H; R⁴=OCH₃; R⁵=H; X¹=X²=Cl; P=TBS (compound 8):

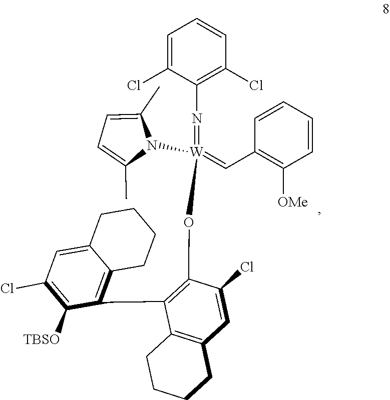

M=W, R¹=2,6-dichlorophenyl; R²=2,5-dimethyl-pyrrol-1-yl; R³=H; R⁴=OCH₃; R5=R6=H; X¹=X²=I; P=TBS (compound 9):

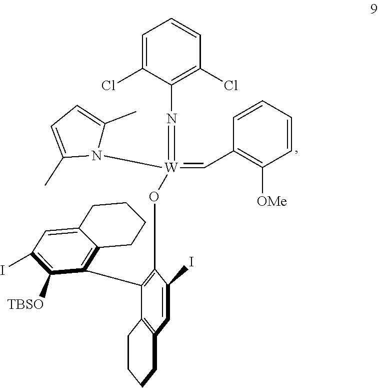

M=W, R¹=2,6-dichlorophenyl; R²=2,5-dimethyl-pyrrol-1-yl; R³=H; R⁴=OCH₃; R⁵=H; X¹=X²=CF₃; P=TBS (compound 10),

M=W, R¹=2,6-dichlorophenyl; R²=2,5-dimethyl-pyrrol-1-yl; R³=H; R⁴=OCH₃; R⁵=H; X¹=X²=C₆F₅; P=TBS (compound 11), and

M=W, R¹=2,6-dichlorophenyl; R²=2,5-dimethyl-pyrrol-1-yl; R³=H; R⁴=OCH₃; R⁵=H; X¹=X²=Br; P=TBS; N=1,10-phenanthroline; n=1 (compound 12):

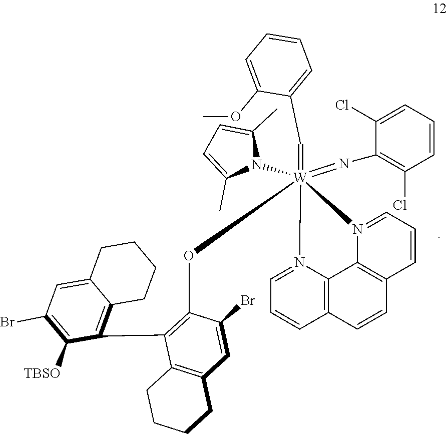

Compound 12 (in which the LO— residue is provided as the R-enantiomer) is characterized by an improved air-stability. It is further characterized in that in solution the complex dissociates upon release of phenanthroline. The remaining alkylidene complex is active in olefin metathesis. This is advantageous in view of known alkylidene-phenanthroline complexes in which the removal of the neutral phenanthroline complex requires the addition of a Lewis acid such as zinc chloride.

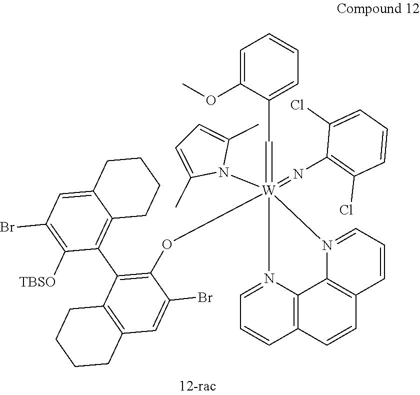

may also be provided in a form wherein LO— is the racemate (or wherein LO— is the S-enantiomer).

In a further aspect regarding the compound of formula (III), R³ may also be C₁₋₅ alkyl, wherein the other residues have the meaning as defined above with respect to said compound of formula (III).

### Compounds Comprising a ═C(phenyl)₂ Moiety wherein the Phenyl Residues Comprise in o-Position a Group Selected from O—(C₁₋₆ Alkyl) and —CH₂—O—(C₁₋₆ Alkyl), Respectively

According to a fourth aspect, the invention relates to a compound of formula (IV)

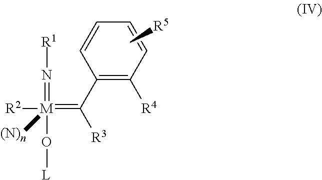

wherein

### M is W;

R¹ is selected from aryl, alkyl and cycloalkyl, each of which is optionally substituted;
R² is pyrrol-1-yl or indol-1-yl, optionally substituted, respectively;

R³ is

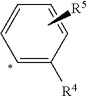

wherein * denotes the bond between R³ and the alkylidene carbon;
R⁴ is selected from O—(C₁-C₆ alkyl), and —CH₂—O—(C₁-C₆ alkyl);

R⁵ is/are one or more residues independently selected from H, C₁-C₆ alkyl, O—(C₁-C₆ alkyl), phenyl, halogen, NO₂, CN, and NHC(O)—(C₁-C₆ alkyl);

### LO— is

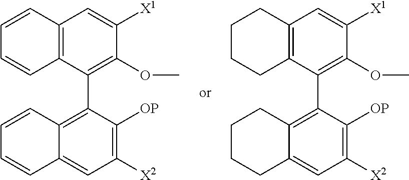

wherein
X¹ and X² are independently selected from halogen, CF₃ and C₆F₅; or
X¹=X²=halogen, CF₃ or C₆F₅;
P is C₁-C₆ alkyl, or a silyl group; and
N is a neutral ligand bound to M, wherein n is 0, 1 or 2,

In a preferred embodiment, R¹ is selected from the group consisting of phenyl substituted with one or more of C₁-C₆ alkyl, O—(C₁-C₆ alkyl), phenyl, halogen and CF₃; t-butyl, and 1-adamantyl.

In one embodiment, R¹ is selected from phenyl substituted with one or more of halogen or CF₃.

In a preferred embodiment, R¹ is 2,6-dichlorophenyl, pentafluorophenly or o-CF₃-C₆H₄.

Preferably, R² is selected from pyrrol-1-yl, 2,5-dimethyl-pyrrol-1-yl, 2,5-diethyl-pyrrol-1-yl, 2,5-diphenyl-pyrrol-1-yl, and indol-1-yl.

In one embodiment, LO— has (R) configuration.

In another embodiment, LO— has (S) configuration

In another embodiment, LO— is racemic.

The use of racemic LO— may be advantageous under economical aspects since racemic LOH is typically cheaper compared to its enantiomers.

The term “silyl” used in connection with P in the OP moiety may be any silyl group forming a covalent bond between silicon and oxygen.

Known groups are e.g. t-butyldimethylsilyl (TBS, TBDMS), trimethylsilyl (TMS), triethylsilyl (TES), triisopropylsilyl (TIPS), t-butyldiphenylsilyl (TBDPS), and triphenylsilyl.

In a preferred embodiment, said neutral ligand N is a nitrile.

Preferably, said nitrile is acetonitrile.

Nitrile binds via N to M.

In another preferred embodiment, said neutral ligand N is a phosphine.

Preferably, said phosphine is selected from the group consisting of dimethylphenyl phosphine, methyldiphenyl phosphine and tris(cyclohexyl) phosphine.

Phosphine binds via P to M.

In a further preferred embodiment, said neutral ligand is a pyridine.

Preferabyl, said pyridine is pyridine as such, or 2,2′-bipyridine, or 1,10-phenanthroline.

Said pyridine may be substituted with one or more substituents independently selected from C₁₋₄ alkyl, C₁₋₄ alkoxy, phenyl, phenoxy and halogen.

Said pyridine binds via N to M, either as a monodentate ligand or a bidentate ligand.

### Metathesis Reactions in the Presence of a Compound of Formula (I), (II), (III), or (IV) or (VI)

According to a fifth aspect, the invention relates to method of performing a metathesis reaction, the method comprising:

performing the metathesis reaction in the presence of a compound of formula (I), (II), (III), or (IV) or (VI) as defined in the first aspect, second aspect, third aspect or fourth aspect, or eighth aspect (defined below), or any embodiment thereof.

In a preferred embodiment, the metathesis reaction is selected from ethenolysis of an internal olefin, cross-metathesis of an olefin, and a ring-closing metathesis reaction.

In a preferred embodiment, the ethenolysis of an internal olefin is the reaction of ethylene with an unsaturated fatty acid ester.

In another preferred embodiment, a cross-metathesis reaction is homo-metathesis of an unsaturated fatty acid ester.

In one embodiment, said unsaturated fatty acid ester is a natural oil.

The term “natural oil” encompasses triglycerides such as vegetable oils, algae oils, fish oils, and animal fats.

In a preferred embodiment, the unsaturated fatty acid ester is the methyl ester (FAME), wherein FAME is selected from methyl oleate, methyl linolate, and methyl linolenoate and mixtures of two or three thereof.

In a particularly preferred embodiment, said unsaturated fatty acid ester is methyl oleate.

Ethenolysis reactions allow for the formation of terminal olefins from internal olefins via a cross-metathesis reaction with ethylene. Efficient ethenolysis of natural products comprising internal olefins such as natural oils or fatty acid methyl esters such as methyl oleate is attractive as a method of obtaining useful chemicals from biomass.

In a further embodiment of the fifth aspect, the metathesis reaction is a ring-dosing metathesis reaction.

### Metathesis Reactions Using Unsaturated Fatty Acid Esters in the Presence of Compound of Formula (V)

According to a sixth aspect, the invention relates to a method of performing a metathesis reaction, wherein the metathesis reaction is ethenolysis of an unsaturated fatty acid ester, a home-metathesis of an unsaturated fatty acid ester, or a ring-dosing reaction, the method comprising:

performing the metathesis reaction in the presence of a compound of formula (V)

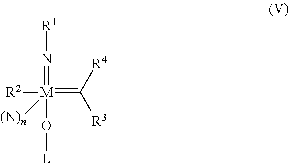

wherein

### M=W;

R¹ is selected from phenyl substituted with one or more of halogen or CF₃;
R² is selected from pyrrol-1-yl or indol-1-yl, optionally substituted, respectively; preferably pyrrol-1-yl, 2,5-dimethyl-pyrrol-1-yl, 2,5-diethyl-pyrrol-1-yl, 2,5-diphenyl-pyrrol-1-yl, and indol-1-yl;
one of R³ and R⁴ is H, and the other is C(CH₃)₂C₆H₅;

### LO— is

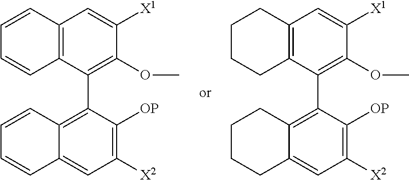

wherein X¹ and X² are independently selected from halogen, CF₃ and C₆F₆; or
X¹=X²=halogen, CF₃ or C₆F₆;
P is C₁-C₆ alkyl, or a silyl group; and
N is a neutral ligand bound to M, wherein n is 0, 1 or 2.

In one embodiment of the sixth aspect, LO— has (R) or (S) configuration; or LO— is racemic.

P and N are defined as in the first aspect.

In a preferred embodiment, in the compound of formula (V) used in the method according to the sixth aspect, R¹ is 2,6-dichlorophenyl, pentafluorophenly or o-CF₃-C₆H₄.

In one embodiment, in the compound of formula (V) used in the method of the sixth aspect

R¹ is 2,6-dichlorophenyl, R² is 2,5-dimethylpyrrol-1-yl, X¹=X²=F; or
R¹ is 2,6-dichlorophenyl, R² is 2,5-dimethylpyrrol-1-yl, X¹=X₂=Cl; or
R¹ is 2,6-dichlorophenyl, R² is 2,5-dimethylpyrrol-1-yl, X¹=X₂=Br; or
R¹ is 2,6-dichlorophenyl, R² is 2,5-dimethylpyrrol-1-yl, X¹=X₂=I.

In one embodiment, the compound of formula (V) is selected from the group consisting of compounds 13,14, 15 and 16:

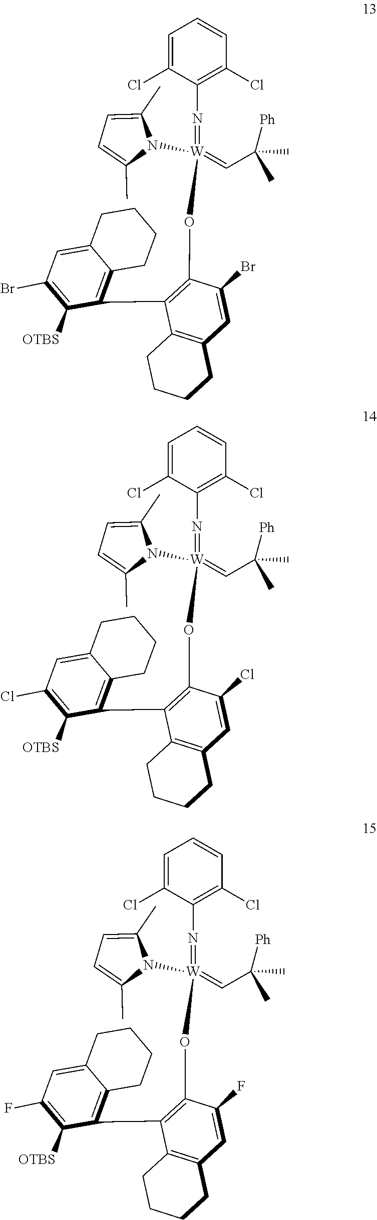

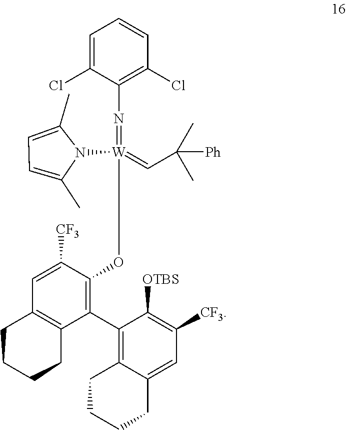

In one embodiment of the sixth aspect, said unsaturated fatty acid ester is a natural oil.

In a preferred embodiment, said unsaturated fatty add ester is a methyl ester (FAME).

In a still more preferred embodiment, the methyl ester is methyl oleate or methyl linolate or methyl linolenoate or a mixture of two or three thereof.

Preferably, the methyl ester is methyl oleate.

In a further embodiment of the sixth aspect, the metathesis reaction is a ring-closing metathesis reaction.

The compounds to be subjected to metathesis may be purified prior to metathesis according to methods known in the art. E.g., suitable methods are described in WO 2014/139679 (XiMo AG).

### Specific ═CHC(Me)₂C₆H₅ Complexes

According to a seventh aspect, the invention relates to a compound of formula 14, 15,16 or 20:

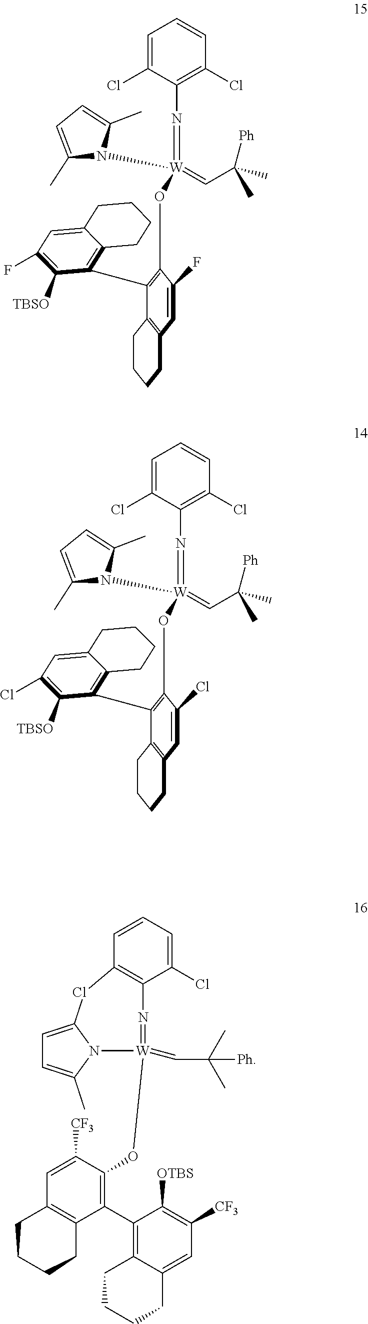

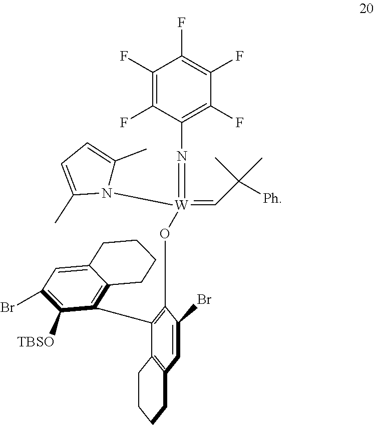

### Compounds Comprising a ═CH(aryl) Moiety (Such as Benzylidene) of Formula (VI)

According to an eighth aspect, the invention relates to a compound of formula (VI)

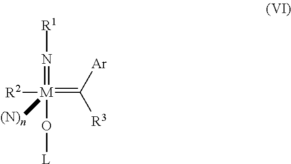

wherein

### M is W;

Ar is selected from phenyl, naphthyl and anthracenyl, optionally substituted, respectively;
R¹ is selected from aryl, alkyl and cycloalkyl, each of which is optionally substituted;
R² is pyrrol-1-yl or indol-1-yl, optionally substituted;
R³ is selected from H;

### LO— is

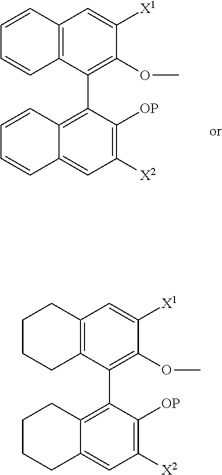

wherein
X¹ and X² are independently selected from halogen, CF₃ and C₆F₅; or
X¹=X²=halogen, CF₃ or C₆F₅;
P is C₁-C₆ alkyl, or a silyl group; and
N is a neutral ligand bound to M, wherein n is 0, 1 or 2,

The term “phenyl, naphthyl and anthracenyl, optionally substituted, respectively” as used herein means that the aryl residue may independently bear (or comprise) one or more of C₁-C₆ alkyl, O—(C₁-C₆ alkyl), phenyl, halogen, NO₂, CN and NHC(O)—(C₁-C₆ alkyl).

In a preferred embodiment, R¹ is selected from the group consisting of phenyl substituted with one or more of C₁-C₆ alkyl, O—(C₁-C₆ alkyl), phenyl, halogen and CF₃; t-butyl, and 1-adamantyl.

In one embodiment, R¹ is selected from phenyl substituted with one or more of halogen or CF₃.

In a preferred embodiment, R¹ is 2,6-dichlorophenyl, pentafluorophenly or o-CF₃-C₆H₄.

Preferably, R² is selected from pyrrol-1-yl, 2,5-dimethyl-pyrrol-1-yl, 2,5-diethyl-pyrrol-1-yl, 2,5-diphenyl-pyrrol-1-yl, and indol-1-yl.

In one embodiment, LO— has (R) configuration.

In another embodiment, LO— has (S) configuration

In another embodiment, LO— is racemic.

The use of racemic LO— may be advantageous under economical aspects since racemic LOH typically is cheaper compared to its enantiomers.

The term “silyl” used in connection with P in the OP moiety may be any silyl group forming a covalent bond between silicon and oxygen.

Known groups are e.g. t-butyldimethylsilyl (TBS, TBDMS), trimethylsilyl (TMS), triethylsilyl (TES), triisopropylsilyl (TIPS), t-butyldiphenylsilyl (TBDPS), and triphenylsilyl.

In a preferred embodiment, said neutral ligand N is a nitrile.

Preferably, said nitrile is acetonitrile.

Nitrile binds via N to M.

In another preferred embodiment, said neutral ligand N is a phosphine.

Preferably, said phosphine is selected from the group consisting of dimethylphenyl phosphine, methyldiphenyl phosphine and tris(cyclohexyl) phosphine.

Phosphine binds via P to M.

In a further preferred embodiment, said neutral ligand is a pyridine.

Preferably, said pyridine is pyridine as such, or 2,2′-bipyridine, or 1,10-phenanthroline.

Said pyridine may be substituted with one or more substituents independently selected from C₁₋₄ alkyl, C₁₋₄ alkoxy, phenyl, phenoxy and halogen.

Said pyridine binds via N to M, either as a monodentate ligand or a bidentate ligand.

In one embodiment of the eighth aspect, the invention relates to a compound of formula (VI-A)

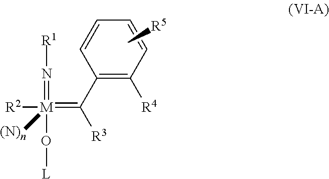

wherein

### M is W;

R¹ is selected from aryl, alkyl and cycloalkyl, each of which is optionally substituted;
R² is pyrrol-1-yl or indol-1-yl, optionally substituted;
R³ is selected from H;
R⁴ is R⁵;
R⁵ is/are one or more independently selected from H, C₁-C₆ alkyl, O—(C₁-C₆ alkyl), phenyl, halogen, NO₂, CN, and NHC(O)—(C₁-C₆ alkyl); wherein O—(C₁-C₆ alkyl) is not in o-position;

### LO— is

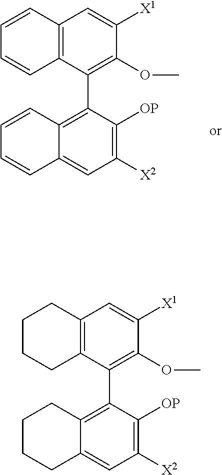

wherein
X¹ and X² are independently selected from halogen, CF₃ and C₆F₅; or
X¹=X²=halogen, CF₃ or C₆F₅;
P is C₁-C₆ alkyl, or a silyl group; and
N is a neutral ligand bound to M, wherein n is 0, 1 or 2.

In a preferred embodiment, R¹ is selected from the group consisting of phenyl substituted with one or more of C₁-C₆ alkyl, O—(C₁-C₆ alkyl), phenyl, halogen and CF₃; t-butyl, and 1-adamantyl.

In one embodiment, R¹ is selected from phenyl substituted with one or more of halogen or CF₃.

In a preferred embodiment, R¹ is 2,6-dichlorophenyl, pentafluorophenly or o-CF₃-C₆H₄.

Preferably, R² is selected from pyrrol-1-yl, 2,5-dimethyl-pyrrol-1-yl, 2,5-diphenyl-pyrrol-1-yl, and indol-1-yl

In one embodiment, LO— has (R) configuration.

In another embodiment, LO— has (S) configuration

In another embodiment, LO— is racemic.

The use of racemic LO— may be advantageous under economical aspects since racemic LOH typically is cheaper compared to its enantiomers.

The term “silyl” used in connection with P in the OP moiety may be any silyl group forming a covalent bond between silicon and oxygen.

Known groups are e.g. t-butyldimethylsilyl (TBS, TBDMS), trimethylsilyl (TMS), triethylsilyl (TES), triisopropylsilyl (TIPS), t-butyldiphenylsilyl (TBDMS), and triphenylsilyl.

In a preferred embodiment, said neutral ligand N is a nitrile.

Preferably, said nitrile is acetonitrile.

Nitrile binds via N to M.

In another preferred embodiment, said neutral ligand N is a phosphine.

Preferably, said phosphine is selected from the group consisting of dimethylphenyl phosphine, methyldiphenyl phosphine and tris(cyclohexyl) phosphine.

Phosphine binds via P to M.

In a further preferred embodiment, said neutral ligand is a pyridine.

Preferably, said pyridine is pyridine as such, or 2,2′-bipyridine, or 1,10-phenanthroline.

Said pyridine may be substituted with one or more substituents independently selected from C₁₋₄ alkyl, C₁₋₄ alkoxy, phenyl, phenoxy and halogen.

Said pyridine binds via N to M, either as a monodentate ligand or a bidentate ligand.

In another embodiment, the invention relates to a compound of formula (VI-B), wherein in the compound of formula (VI) Ar=naphthyl, optionally substituted.

In one embodiment, the compound is of formula (VI-Ba), wherein naphthyl is naphth-1-yl, optionally substituted.

In another embodiment, the compound is of form a (VI-Bb), wherein naphthyl is naphth-2-yl, optionally substituted.

In one embodiment, the compound is of formula (VI-C), wherein in the compound of formula (VI) Ar=anthracenyl, optionally substituted.

In one embodiment, the compound is of formula (VI-Ca), wherein anthracenyl is anthracen-9-yl, optionally substituted.

In another embodiment, the compound is of formula (VI-Cb), wherein anthracenyl is anthracen-1-yl, optionally substituted.

In another embodiment, the compound is of formula (VI-Cc), wherein anthracenyl is anthracen-2-yl, optionally substituted.

The compounds of formula (VI) may also be used in the metathesis reaction as defined in the fifth aspect.

In another aspect, the invention relates to a composition comprising a compound of formula (I), (II), (III), (IV), (V) or (VI) and an olefin to be metathesized, wherein the olefin to be metathesized has been subjected to a trialkyl aluminium compound prior to metathesis.

In a preferred embodiment, in the compound of formula (I), (II), (III), (IV), (V) or (VI), LO— is racemic.

### EXAMPLES

3,3′-disubstituted 1,1-binaphthyl-diol (binol) derivatives and 3,3′-disubstituted 5,5′,6,6′,7,7′,8,8′-octahydro-1,1-binaphthyl-diol (bitet) derivatives were synthesized according to known methods, e.g. as reported by E. S. Sattely et al., J. Am. Chem. 2009, 131, 943-953.

### Example 1 (Comparative Example)

### Synthesis of WNAr^{Cl}(Me₂Pyrr)((R)-Br-TBSOBitetO)(CH(Me)₂Ph) (Compound 4)

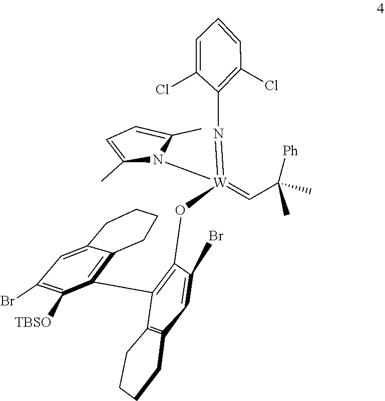

Stock solutions (c=0.1 M, in benzene-d6) were prepared from both the bispyrrolide precursor (WNAr^{Cl}(Me₂Pyrr)₂(CHCMe₂Ph)) and from (R)-3,3′-substituted-2′-(tert-butyldimethylsilyloxy)-5,5′,6,6′,7,7′,8,8′-octahydro-1,1′- binaphthyl-2-ol. 100 μl of the stock solutions was mixed and stirred overnight at rt. Then 500 μl benzene-d6 was added and the sample was measured by ¹H NMR 300 MHz. The solution was used in catalytic reactions without further transformation.

Major diastereomer, 1H-NMR (C₆D₆ ref ¹H solvent=7.16 ppm): −0.06 (s, 3H), 0.11 (s, 3H), 0.93 (s, 3H), 1.25-1.60 (m br, 8H), 1.69 (s, H), 1.73 (s, H), 2.26 (br, 6H), 2.00-2.60 (m, 4H), 5.97 (br, 2H), 6.23 (t, 1H, 3JHH=8.1 Hz), 6.85 (d, 2H, 3JHH=8.1 Hz), 6.93 (m, 1H), 7.09 (m, 2H), 7.16 (s, 1H), 7.24 (s, 1H), 7.42 (m, 2H), 9.73 (s, 1H, 1JCH_syn=117.8 Hz, 2JWH=16.0 Hz) ppm.

### Example 2

### Synthesis of WNAr^{Cl}(CHCMe₂Ph)(Me₂Pyr)((R)-Br-TBSBitet-O))(MeCN) (Compound 1)

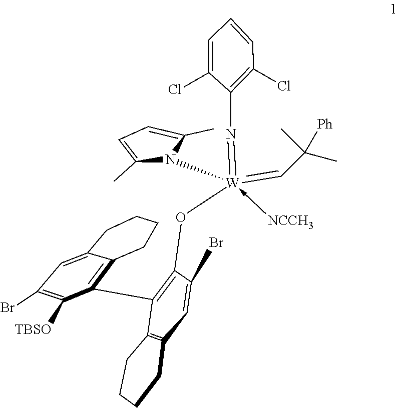

The reaction was carried out in a N₂ filled glovebox. A round-bottomed flask was equipped with a magnetic stirring bar. The flask was charged with the starting W(NArCl)(CHCMe₂Ph)(2,5-Me₂Pyr)₂ complex (0.20 g, 0.30 mmol), then it was mixed with toluene (6 mL) resulting in a brownish yellow homogenous solution. Then the ligand (R)-3,3′-substituted-2′-(tert-butyldimethylsilyloxy)-5,5′,6,67,7′,8,8′-octahydro-1,1′-binaphthyl-2-ol, 0.17 g, 0.30 mmol) was added as a solid to the solution at ambient temperature. The reaction mixture was stirred overnight; the progress of the reaction was monitored by NMR. The solvent was removed under reduced pressure. The residue was dissolved in n-pentane (3 mL) resulting in orange-red homogenous solution. To this solution MeCN (18.5 mg, 24 μL, 0.45 mmol) was added at rt. Upon addition of the MeCN yellowish precipitate crashed out of the solution. The mixture was placed into the glovebox's fridge for a day. The yellow precipitate was filtered out, washed with cold n-pentane (3 mL) and dried under reduced pressure yielding the product as yellow powder (m=217 mg, 62%).

¹H NMR (C₆D₆, 300 MHz): δ 9.85 ppm (s, 1H, CHCMe2Ph), 7.41 (d, 2H, aromatic), 7.25 (s, 1H, aromatic), 7.16 (s, 1H, aromatic), 7.08 (t, 2H, aromatic), 6.92 (t, 1H, aromatic), 6.85 (d, 2H, aromatic), 6.22 (t, 1H, aromatic), 5.99 (br s, 2H, NC4H2), 2.27 (s, 6H, Me₂NC₄H₂), 2.15 (m, 8H, Bitet), 1.72 (s, 3H, PhCMe₂), 1.70 (s, 3H, PhCMe₂), 1.39 (m, 8H, Bitet), 0.93 (s, 9H, TBS), 0.59 (s, 3H, MeCN), 0.14 (s, 3H, TBS), −0.10 (s, 3H, TBS).

### Example 3

### Synthesis of W(NAr^{Cl})(CHCMe₂Ph)(Me₂Pyr)((R)-Br-TBSOBitet-O)(Py) (Compound 3)

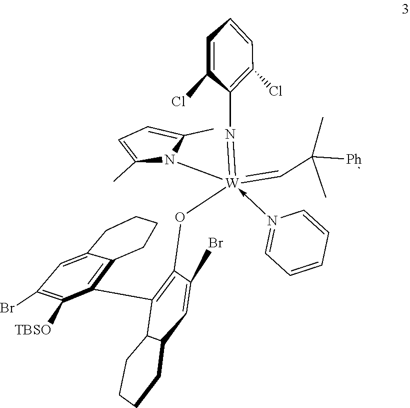

The reaction was carried out in a N₂ filled glovebox. A round-bottomed flask was equipped with a magnetic stirring bar. The flask was charged with the starting W(NAr^{Cl})(CHCMe₂Ph)(Me₂Pyr)₂ complex (0.20 g, 0.30 mmol), then it was mixed with toluene (6 mL) resulting in a brownish yellow homogenous solution. Then the ligand (R)-3,3′-substituted-2′-(tert-butyldimethylsilyloxy)-5,5′,6,6′,7,7′,8,8′-octahydro-1,1′-binaphthyl-2-ol, 0.17 g, 0.30 mmol) was added as a solid to the solution at ambient temperature. The reaction mixture was stirred overnight, the progress of the reaction was monitored by NMR. The solvent was removed under reduced pressure. The residue was dissolved in n-pentane (3 mL) resulting in orange-red homogenous solution. To this solution few drops of pyridine was added at rt. Upon addition of the pyridine yellowish precipitate crashed out of the solution. The mixture was placed into the glovebox's fridge for a day. The yellow precipitate was filtered out, washed with cold n-pentane (3 mL) and dried under reduced pressure yielding the product as yellow powder (m=227 mg, 62%).

¹H NMR (toluene-d₈, 300 MHz, 70° C.): δ 9.63 ppm (s, 1H, CHCMe₂Ph), 8.45 (d, 2H, aromatic), 7.34 (d, 2H, aromatic), 7.15 (s, 2H, aromatic), 7.06-6.86 (m, aromatic), 6.69 (m, 2H, aromatic), 6.32 (t, 1H, aromatic), 5.81 (br s, 2H, NC₄H₂), 2.36 (m, 8H, Bitet), 2.16 (s, 6H, Me2NC₄H₂), 1.68 (s, 3H, PhCMe₂), 1.65 (s, 3H, PhCMe₂), 1.42 (m, 8H, Bitet), 0.84 (s, 9H, TBS), 0.03 (s, 3H, TBS), −0.06 (s, 3H, TBS).

### Example 4

### Synthesis of WNAr^{Cl}(Me₂Pyrr)(R)-TBSBinol)(CHCMe₂Ph) (Compound 5)

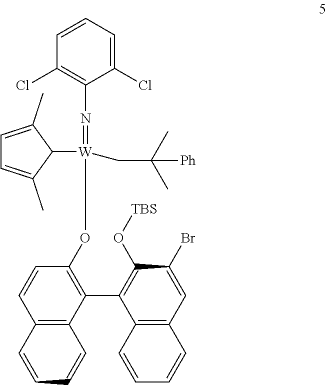

Bispyrrolide precursor, WNAr^{Cl}(Me₂Pyrr)2(CHCMe₂Ph) (0.035 mmol, 23.3 mg) was dissolved in benzene-d6 (0.35 mL). ((R)-3,3′-Dibromo-2′-(tert-butyldimethylsilyloxy)-1,1′-binaphthyl-2-ol (0.035 mmol, 19.5 mg) was dissolved in benzene-d6 (0.35 mL) and added to the bispyrrolide precursor at r.t. The mixture was stirred at r.t. overnight. After ¹H NMR measurement to confirm structure the catalyst solution was used without further transformation,

### Example 5

### Synthesis of WNAr^{Cl}(Me₂Pyrr)((R)-I-TBSOBitetO)(CH(2-MeO-C₆H₄)) (Compound 9)

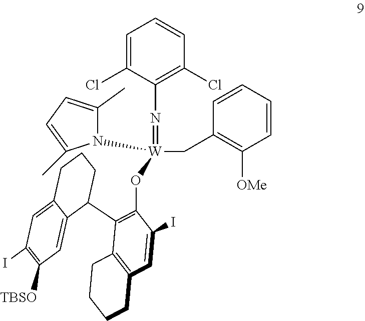

WNAr^{Cl}(Me₂Pyrr)((R)-I-BITET-O)(CHCMe₂Ph), (0.15 mmol, 191 mg) was dissolved in toluene (2 mL) and 2-MeO-styrene (0.165 mmol, 22.1 mg) was added to it. The mixture was stirred for 1 day at r.t. Additional amount of 2-MeO-styrene 0.1 mmol (14 mg) was added, and the mixture was further stirred at r.t. Then the mixture was evaporated to dry, triturated with pentane (4 mL), then with acetonitrile and cooled to −40° C. overnight. The solid was isolated by filtration and washed with acetonitrile. Isolated yield: 16 mg, 8.7%.

¹H NMR (C₆D₆, δ_{ref 1H solvent}=7.16 ppm, 25° C., 300 MHz): 11.28 ppm, characteristic alkylidene signal.

### Example 6

### Synthesis of WNAr^{Cl}(Me₂Pyrr)((R)-F-Bitet-O)(CHCMe₂Ph) (Compound 7)

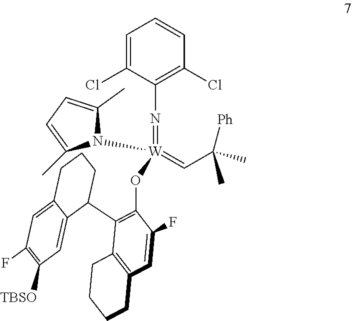

Bispyrrolide precursor, WNAr^{Cl}(Me₂Pyrr)₂(CHCMe₂Ph) (0.5 mmol, 332 mg) was dissolved in toluene (1 mL), (R)-3,3′-fluoro-2′-(tert-butyldimethylsilyloxy)-5,5′,6,6′,7,7′,8,8′-octahydro-1,1′-binaphthyl-2-ol (0.5 mmol, 222 mg) was dissolved in toluene (2 mL) and added to the bispyrrolide precursor at r.t. The mixture was stirred at r.t. overnight then evaporated to dry, triturated with acetonitrile (3 mL) and cooled to −40° C. overnight. The solid was filtered off and washed with acetonitrile. Then dissolved in benzene and evaporated to dry to remove acetonitrile. isolated yield: 229 mg, 45%.

¹H-NMR (C₆D₆, δ_{ref 1H solvent}=7.16 ppm, 25° C., 300 MHz): 9.53 ppm; characteristic alkylidene signals.

¹⁹F-NMR (C₆D₆, δ_{ref 1H solvent}=7.16 ppm, 25° C., 282.4 MHz): −134.8 (s, 1F), −133.2 (s, 1F).

### Example 7Synthesis of WNAr^{Cl}(Me₂Pyrr)(CHMe₂Ph)(Cl-(R)-TBSOBitet-O) (Compound 14)

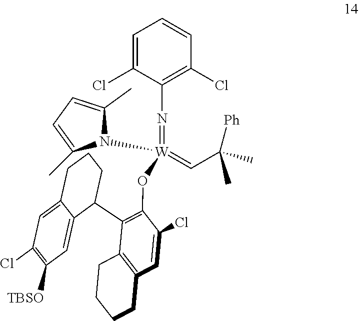

Bispyrrolide precursor, WNAr^{Cl}(Me₂Pyrr)₂(CHCMe₂Ph) (0.5 mmol, 332 mg) was dissolved in toluene (1 mL). (R)-3,3′-chloro-2′-(tert-butyldimethylsilyloxy)-5,5′,6,6′,7,7′,8,8′-octahydro-1,1′-binaphthyl-2-ol (0.5 mmol, 239 mg) was dissolved in toluene (2 mL) and added to the bispyrrolide precursor at r.t. The mixture was stirred at r.t. overnight then evaporated to dry, triturated with acetonitrile (2 mL) and cooled to −40° C. overnight. The solid was filtered off and washed with acetonitrile. Isolated yield: 287 mg, 54.8%.

¹H-NMR (C₆D₆, δ_{ref 1H solvent}=7.16 ppm, 25° C., 300 MHz) 9.83 ppm, characteristic alkylidene signals.

### Example 8

The compounds according to the invention were tested in a homo-metathesis reaction of methyl 9-decenoate (9-DAME):

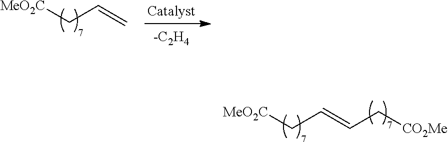

The substrate was purified by an adsorption method or triethylaluminum (TEAl) treatment according to methods known from WO2014/139679 (XiMo). Reactions in which the substrate was purified by an adsorptive method were terminated after 24 hours, and reactions in which the substrate was purified by means of triethylaluminum were terminated after 4 hours (rt=room temperature)

Conversion and formed E- and Z-isomers were determined via chromatography. The results are shown in Table 1:

TABLE 1

| | Purification | Loading | | | |
|---|---|---|---|---|---|
| | of | [ppm/mole | T | Conversion | E/Z |
| Compound | 9-DAME | 9-DAME] | [° C.] | [%[ | ratio |
| 6 | adsorptive | 25 | rt | 76 | 3/97 |
| 7 | adsorptive | 25 | rt | 76 | 17/83 |
| 8 | adsorptive | 25 | rt | 45 | 3/97 |
| 6 | adsorptive | 25 | rt | 76 | 3/97 |
| 9 | adsorptive | 25 | rt | 14 | 1/99 |
| 6 | adsorptive | 50 | rt | 77 | 21/79 |
| 12 | adsorptive | 50 | 50 | 77 | 9/91 |
| 6 | adsorptive | 50 | rt | 90 | 9/91 |
| 4 | adsorptive | 50 | rt | 89 | 7/93 |
| 1 | adsorptive | 50 | rt | 88 | 6/94 |
| 6 | TEAI | 25 | rt | 78 | 4/96 |
| 4 | TEAI | 25 | rt | 84 | 3/97 |
| 1 | TEAI | 25 | rt | 82 | 4/96 |
| 3 | TEAI | 25 | rt | 26 | 2/98 |
| 12 | TEAI | 25 | rt | 77 | 4/96 |
| 15 | TEAI | 25 | rt | 72 | 3/97 |
| 14 | TEAI | 25 | rt | 73 | 4/96 |
| 2 | TEAI | 25 | rt | 70 | 2/98 |
| 16 | TEAI | 25 | rt | 54 | 2/98 |
| 7 | TEAI | 25 | rt | 60 | 14/86 |
| 8 | TEAI | 25 | rt | 58 | 3/97 |
| 9 | TEAI | 25 | rt | 70 | 2/98 |

### Example 9

The compounds according to the invention were tested in a ring-closing metathesis reaction:

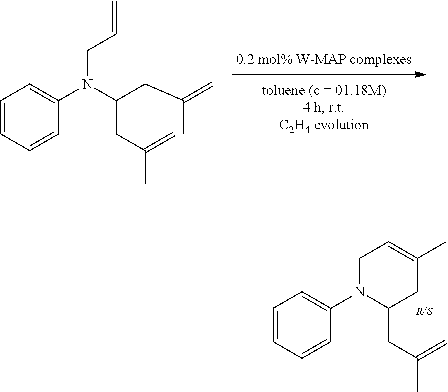 [SMILES: C=CCN(c1ccccc1)C(CC(=C)C)CC(=C)C ; C=C(C)CC1CC(C)=CCN1c1ccccc1]

Under the atmosphere of the glovebox in an oven-dried 4 mL vial, the substrate was added by an automatic pipette and its weight was precisely measured. It was dissolved in 1 mL toluene, then the catalyst stock was added to it. The vial was closed by a perforated cap and the reaction mixture was stirred at r.t. for 4 h. Then 1 mL MeOH was added to the sample to quench the catalyst. A 20 mL plastic syringe was filled with 0.5 mL silica layer and 1 mL of the reaction mixture was filtered through it and washed with 20 mL ethyl acetate. The sample was analysed by GCMS to determine conversion. Enantiomer ratio of the product was determined by chiral HPLC (Agilent 1200 Plus HPLC, with diode array detector at 256 nm. Column: Kromasil 5-AmyCoat 4.6×150 mm, using H₂O-MeOH gradient elution).

The results are shown in Table 2:

TABLE 2

| | | | Product | |
|---|---|---|---|---|
| | | | in | |
| | Loading | | reaction | |
| | catalyst | Conversion | mixture | |
| Compound | [pppmmole] | [%] | [%] | S:R |
| 4 | 2000 | 100 | 96.2 | 5:95 |
| 2 | 2000 | 100 | 96.2 | 3:97 |
| 14 | 2000 | 100 | 97.1 | 7:93 |

### Example 10

Compounds according to the invention were tested in ethenolysis of methyl oleate.

The substrate was purified using triethylaluminum (TEAl) according to methods known from WO 2014/139679 (XiMo). Methyl oleate was mixed with 700 ppmwt TEAl and the mixture was stirred at room temperature for 4 hours.

In a nitrogen gas filled glovebox, fatty acid methyl ester was measured into 30 mL glass vials and mixed with the stock solution of triethylaluminum (23% wt in toluene). The optimal triethylaluminum amount was determined previously and was found to be 700 ppm. Mixtures were stirred at r.t. for 1 hour. Catalysts were added as a stock solution (0.01 M in benzene) The vial was placed into a stainless steel autoclave equipped with an alublock and was stirred at 50° C. under 10 atm of ethylene gas overpressure for 18 hours. Ave reactions were performed in the same autoclave with common gas space. The excess of ethylene was let out. From the reaction mixture 2.0 μl was taken out and diluted to 1.5 ml with n-pentane and analyzed by GCMS-FID, (Shimadzu 2010 Plus, column: Zebron ZB-35HT INFERNO, 30 m×0.25 mm×0.25 μm.

*reaction with 12-rac was prepared in 250 mL scale as the catalyst was portioned to it as a powder due to its insolubilityQuantification of the liquid phase by GC indicated the conversion given in Table 3 below:

TABLE 3

| | | | Molar | | 9- | | |
|---|---|---|---|---|---|---|---|
| Com- | | | ratio | Total | DAME | | |
| pound | ppm | ppm | Substrate/ | conversion | yield | 9-DAME | |
| No | wt | mole | catalyst | [%] | [%] | selectivity | TON |
| 1 | 100 | 25 | 40116 | 90 | 78 | 0.9 | 31130 |
| 1 | 50 | 12 | 80232 | 76 | 64 | 0.8 | 51188 |
| 6 | 50 | 13 | 76610 | 80 | 68 | 0.8 | 51712 |
| 6-rac ¹ | 50 | 13 | 76610 | 78 | 66 | 0.8 | 50409 |
| 6-et₂py ² | 100 | 25 | 39262 | 91 | 78 | 0.9 | 30585 |
| 6-et₂py | 50 | 13 | 78523 | 75 | 61 | 0.8 | 47899 |
| 12 | 100 | 22 | 44449 | 87 | 76 | 0.9 | 33603 |
| 12 | 50 | 11 | 88898 | 76 | 63 | 0.8 | 56273 |
| 4 | 100 | 26 | 38716 | 74 | 61 | 0.8 | 23655 |
| 12-rac ³ | 50 | 11 | 88898 | 70 | 58 | 0.8 | 51650 |
| 4 | 50 | 13 | 77431 | 55 | 43 | 0.8 | 33528 |

¹ Compound 6 wherein LO is racemic
² Compound 6 wherein 2,5-dimethyl-pyrrol-1-yl has been replaced by 2,5-diethyl-pyrrol-1-yl
³ Compound 12 wherein LO is racemic

Under comparable conditions, the ethenolysis of methyl oleate using the Mo analog of compound 4 resulted in a yield of 9-DAME of around 30% and a total conversion of around 40%.

### Example 11

### WNAr^{Cl}(CHCMe₂Ph)(Me₂Pyr)((S)-Br-TBSBitet-O))(MeCN)

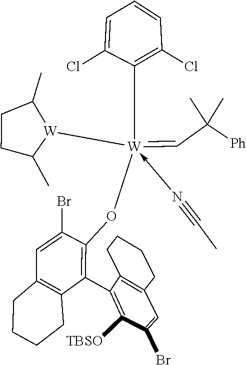

To bispyrrolide W(NAr-2,6-diCl)(CHAr-o-OMe)(2,5-Me₂Pyr)₂ (1000 mg, 1.51 mmol) dissolved in toluene (30 mL), ((S)-3,3′-Dibromo-2′-(tert-butyldimethylsilyloxy)-1,1′-binaphthyl-2-ol (853 mg, 1.51 mmol) was added as a solid slowly, portion-wise, at room temperature. The reaction mixture was stirred at room temperature overnight. Complete conversion into the 14-electron MAP complex was confirmed by NMR analysis prior to the work-up. The solvent was evaporated in vacuum. The residue was triturated in CH₃CN, yielding the title compound as a yellow powder. Yield: 360 mg (20%).

¹H NMR (C6D6, 300 MHz): δ 9.89 ppm (s, 1H, CHCMe2Ph) characteristic alkylidene signal.

### Example 12

### WNAr^{Cl}(CHCMe₂Ph)(Me₂Pyr)((rac)-Br-TBSBitet-O))(MeCN)

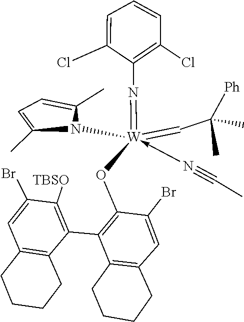

The reaction was carried out in a N₂ filled glovebox. A 100 mL flask was charged with the starting W(NAr-2,6-diCl)(CHCMe₂Ph)(2,5-Me₂Pyr)₂ complex (1.00 g, 1.51 mmol), then it was mixed with toluene (30 mL) resulting in a brownish yellow homogenous solution. Then the ligand ((Rac)-3,3′-Dibromo-2′-(tert-butyldimethylsilyloxy)-1,1′-binaphthyl-2-ol, 0.853 g, 1.51 mmol) was added as a solid to the solution at ambient temperature. The reaction mixture was stirred overnight, the progress of the reaction was monitored by NMR. The solvent was removed under reduced pressure. The residue was triturated in acetonitrile resulting in a yellow precipitate. The yellow precipitate was filtered out, washed with cold n-pentane (10 mL) and dried under reduced pressure yielding the product as yellow powder (m=1102 mg, 62%).

¹H NMR (C₆D₆, 300 MHz): δ 9.92 ppm (s, 1H, CHCMe₂Ph) characteristic alkylidene signal.

### Example 13

### Synthesis of WNAr^{Cl}(Me₂Pyrr)((R)-Br-TBSOBitet-O)(CH(2-MeOC₆H₄)) (Compound 6)

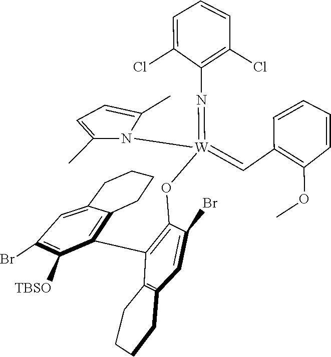

2-Methoxystyrene (1.19 g, 8.85 mmol) was dissolved in toluene and to a previously in-situ prepared solution of W(NAr^{Cl})(Me₂Pyrr)((R)-Br-TBSOBitetO)(CH(Me)₂Ph) (7.63 mmol as a sol in toluene 0.1 mol/L) the mixture was stirred for a weekend at r.t. The completion of the reaction was monitored by ¹H NMR. The reaction mixture was evaporated under reduced pressure to dry. The deep red brown residue was mixed with pentane (20 mL) and red precipitate crashed out of the solution. The precipitate was filtered out, washed with pentane and dried. Isolated yield: 5624 mg, 65%.

¹H-NMR (C₆D₆; δ_{ref 1H solvent}=7.16 ppm): −0.24 (s, 3H, CH₃ TBS), 0.24 (s, 3H, CH₃ TBS), 0.88 (s, 3H, C(CH₃)₃ TBS), 1.28-1.53 (m br, 8H, C6-H₂, C6′-H₂, C7-H₂, C7′-H₂ BITET), 1,76 (br, 3H, CH₃ diMe-pyrrol), 1.86-2.40 (m, 4H, C8-H₂, C8′-H₂ BITET), 2.06, 2.11 (m, 2H, C5-H₂ BITET), 2.47 (br, 2H, C5′-H₂ BITET), 3.16 (br, 3H, CH₃ diMe-pyrrol), 3.67 (s, 3H, MeObenzylidene CH₃), 6.15 (br, 2H, CₐᵣH diMe-pyrrol), 6.20 (t, 1H, ³J_{HH}=8.1, Hz N—Ar Cₚₐᵣₐ-H) 6.23 (dd, 1H, J=7.4, 1.2 Hz, MeObenzylidene C6-H), 6.48 (ddd, 1H, J=8.1,7.4, 1.2 Hz, MeObenzylidene C4-H), 6.64 (d, 1H, J=8.1 Hz, MeObenzylidene C3-H), 6.68 (s, 1H, C3-H BITET), 6.84 (td, 1H, J=7.4, 1 Hz, MeObenzylidene C5-H), 6.84 (d, 2H, ³J_{HH}=8.1 Hz, N—Ar Cₘₑₜₐ-H), 7.28 (s, 1H, C3′-H BITET), 11.28 (s, 1H, W═CH, ¹J_{CH_anti}=155 Hz, ²J_{WH}=7.2 Hz) ppm.

### Synthesis of WNAr^{Cl}(Me²Pyrr)((S)-Br-TBSOBitet-O)(CH(2-MeOC₆H₄))

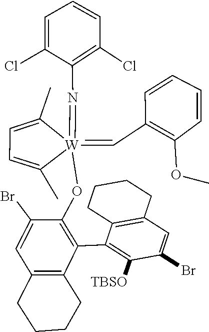

Bispyrrolide precursor, W(NAr^{Cl})(Me₂Pyr)₂(CHCMe₂Ph) (1 mmol, 664 mg) was dissolved in benzene (2 mL). (S)-3,3′-dibromo-2′-(tert-butyldimethylsilyloxy)-5,5′,6,6′,7,7′,8,8′-octahydro-1,1′-binaphthyl-2-ol (1 mmol, 566 mg) was dissolved in benzene (2 mL) and added to the bispyrrolide precursor at rt. The reaction was monitored by ¹H NMR. The mixture was stirred at r.t. overnight then evaporated to dry. The residue was redissolved in 4 mL benzene and 2-MeO-styrene (1.5 mmol, 201 mg) was added and the mixture was stirred overnight at r.t. Then the mixture was evaporated to dry, triturated with n-pentane (5 mL) and cooled to −40° C. overnight. The solid was isolated by filtration and washed with n-pentane (3 mL). Red-brown solid (500 mg, yield 44%) was obtained.

¹H NMR (C₆D₆, 300 MHz): δ 11.28 ppm (s, 1H, CHCMe₂Ph) characteristic alkylidene signal.

### Synthesis of WNAr^{Cl}(Me₂Pyrr)((rac)-Br-TBSOBitet-O)(CH(2-MeOC₆H₄))

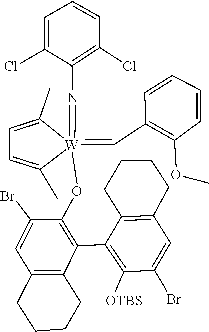

The reaction was carried out in a N₂ filled glovebox. A round-bottomed flask was equipped with a magnetic stirring bar. The flask was charged with the starting W(NAr-2,6-diCl)(CHCMe₂Ph)(2,5-Me₂Pyr)₂ complex (359 mg, 0.54 mmol), then it was mixed with toluene (10.5 mL) resulting in a brownish yellow homogenous solution. Then the ligand ((Rac)-3,3′-Dibromo-2′-(tert-butyldimethylsilyloxy)-1,1″-binaphthyl-2-ol, 0.296 g, 0.524 mmol) was added as a solid to the solution at ambient temperature. The reaction mixture was stirred overnight, the progress of the reaction was monitored by NMR. The solvent was removed under reduced pressure. The residue was dissolved in n-pentane (4 mL), the solids were removed by filtration, the filtrate was concentrated to dryness. The residue was dissolved in toluene (6 mL) and 2-methoxy styrene (0.594 mmol, 80 mg) was added. The reaction mixture was stirred overnight, the solvent was evaporated under reduced pressure to dryness. The deep red residue was taken up in dry n-pentane (ca. 5 mL), and the resulting red crystalline solid was isolated by filtration, washed with n-pentane and dried in vacuum. (m=299 mg, 49%).

¹H NMR (C₆D₆, 300 MHz): δ 9.92 ppm (s, 1H, CHCMe₂Ph) characteristic alkylidene signal.

1. Compound of formula (I) 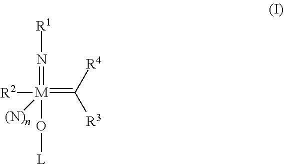
wherein
M=W;
R¹ is selected from phenyl substituted with one or more of halogen or CF₃;
R² is selected from pyrrol-1-yl or indol-1-yl, optionally substituted, respectively;
one of R³ and R⁴ is H, and the other is C(CH₃)₂C₆H₅;
LO— is 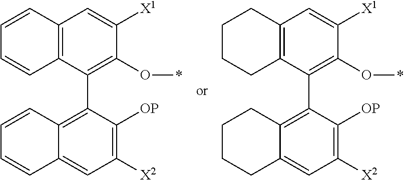
wherein X¹ and X² are independently selected from halogen, CF₃ and C₆F₆; or
X¹=X²=halogen, CF₃ or C₆F₅;
P is C₁-C₆ alkyl, or a silyl group; and
N is a neutral ligand bound to M,
wherein n is 1 or 2, when LO— is a O-bitet ligand, or
wherein n is 0, 1, or 2, when LO— is a O-binol ligand.

2. Compound of formula (II) 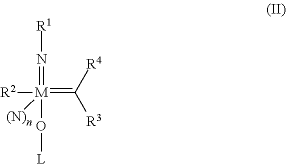
wherein
M=W;
R¹ is selected from aryl, alkyl and cycloalkyl, each of which is optionally substituted;
R² is selected from pyrrol-1-yl or indol-1-yl, optionally substituted, respectively;
one of R³ and R⁴ is H, and the other is C(CH₃)₂C₆H₅; wherein the phenyl group of the C(CH₃)₂C₆H₅ moiety is additionally substituted in o-position with a group selected from O—(C₁-C₆ alkyl) and —CH₂—O—(C₁-C₆ alkyl);
LO— is 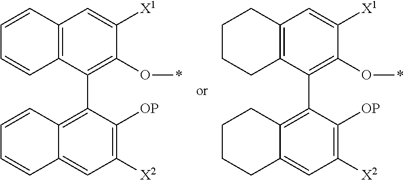
wherein X¹ and X² are independently selected from halogen, CF₃ and C₆F₆; or
X¹=X²=halogen, CF₃ or C₆F₅;
P is C₁-C₆ alkyl, or a silyl group; and
N is a neutral ligand bound to M, wherein n is 0, 1 or 2.

3. Compound of formula (III) 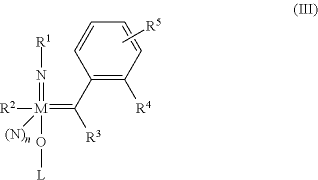
wherein
M is W;
R¹ is selected from aryl, alkyl and cycloalkyl, each of which is optionally substituted;
R² is pyrrol-1-yl or indol-1-yl, optionally substituted, respectively;
R³ is H;
R⁴ is selected from O—(C₁-C₆ alkyl), and —CH₂—O—(C₁-C₆ alkyl);
R⁵ is/are one or more independently selected from H, C₁-C₆ alkyl, O—(C₁-C₆ alkyl), phenyl, halogen, NO₂, CN, and NHC(O)—(C₁-C₆ alkyl);
LO— is 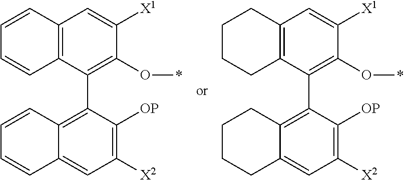
wherein
X¹ and X² are independently selected from halogen, CF₃and C₅F₅; or
X¹=X²=halogen, CF₃ or C₆F₅;
P is C₁-C₆ alkyl, or a silyl group; and
N is a neutral ligand bound to M, wherein n is 0, 1 or 2.
under the proviso that a compound of formula 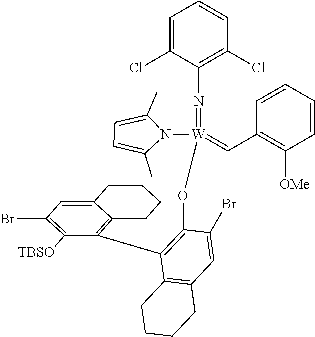
is excluded.

4. Compound of formula (V) 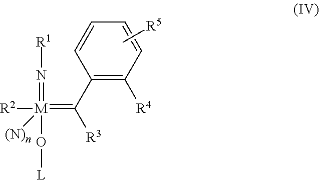
wherein
M is W;
R¹ is selected from aryl, alkyl and cycloalkyl, each of which is optionally substituted;
R² is pyrrol-1-yl or indol-1-yl, optionally substituted, respectively;
R³ is 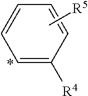
wherein * denotes the bond between R³ and the alkylidene carbon;
R⁴ is selected from O—(C₁-C₆ alkyl), and —CH₂—O—(C₁-C₆ alkyl);
R⁵ is/are one or more independently selected from H, C₁-C₆ alkyl, O—(C₁-C₆ alkyl), phenyl, halogen, NO₂, CN, and NHC(O)—(C₁-C₆ alkyl);
LO— is 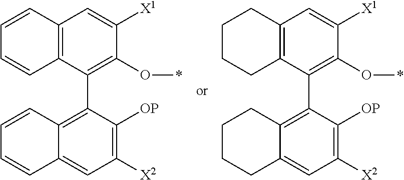
wherein
X¹ and X² are independently selected from halogen, CF₃ and C₆F₅; or
X¹=X²=halogen, CF₃ or C₆F₅;
P is C₁-C₆ alkyl, or a silyl group; and
N is a neutral ligand bound to M, wherein n is 0, 1 or 2.

5. Compound of formula (VI) 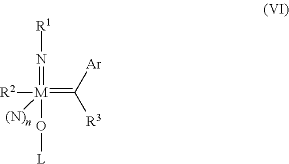
wherein
M is W;
Ar is selected from phenyl, naphthyl and anthracenyl, optionally substituted, respectively;
R¹ is selected from aryl, alkyl and cycloalkyl, each of which is optionally substituted;
R² is pyrrol-1-yl or indol-1-yl, optionally substituted, respectively;
R³ is selected from H;
LO— is 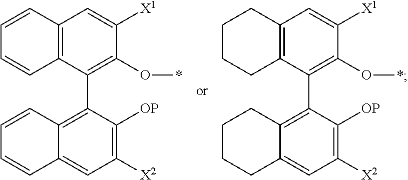
wherein
X¹ and X² are independently selected from halogen, CF₃ and C₆F₅; or
X¹=X²=halogen, CF₃ or C₈F₅;
P is C₁-C₆ alkyl, or a silyl group; and
N is a neutral ligand bound to M, wherein n is 0, 1 or 2;
preferably wherein, when the compound of formula (VI) is a compound of formula (VI-A), 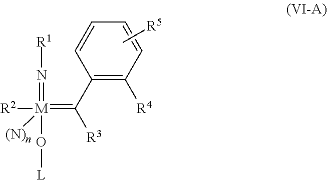
R⁴ is R⁵; and
R⁵ is/are one or more independently selected from H, C₁-C₆ alkyl, O—(C₁-C₆ alkyl), phenyl, halogen, NO₂, CN, and NHC(O)—(C₁-C₆ alkyl); wherein O—(C₁-C₆ alkyl) is not in o-position.

6. Compound of any one of claims 2 to 5, wherein R¹ is selected from the group consisting of phenyl substituted with one or more of C₁-C₆ alkyl, O—(C₁-C₆ alkyl), phenyl, halogen and CF₃; t-butyl, and 1-adamantyl.

7. Compound of any one of the preceding claims, wherein R¹ is selected from 2,6-dichlorophenyl, pentafluorophenyl and o-trifluoromethylphenyl.

8. Compound of any one of the preceding claims, wherein R² is selected from pyrrol-1-yl, 2,5-dimethyl-pyrrol-1-yl, 2,5diethyl-pyrrol-1-yl, 2,5-diphenyl-pyrrol-1-yl, and indol-1-yl.

9. Compound of any one of the preceding claims, wherein
LO— has (R) configuration; or
LO— has (S) configuration; or
LO is racemic.

10. Compound of claim 3,
wherein
M=W, R¹=2,6-dichlorophenyl; R²=2,5-dimethyl-pyrrol-1-yl; R³=H; R⁴=OCH₃; R⁵=H; X¹=X²=F;
M=W, R¹=2,6-dichlorophenyl; R²=2,5-dimethyl-pyrrol-1-yl; R³=H; R⁴=OCH₃; R⁵=H; X¹=X²=Cl;
M=W, R¹=2,6-dichlorophenyl; R²=2,5-dimethyl-pyrrol-1-yl; R³=H; R⁴=OCH₃; R⁵=R⁶=H; X¹=X²=l;
M=W, R¹=2,6-dichlorophenyl; R²=2,5-dimethyl-pyrrol-1-yl; R³=H; R⁴=OCH₃; R⁵=H; X¹=X²=CF₃;
M=W, R¹=2,6-dichlorophenyl; R²=2,5-dimethyl-pyrrol-1-yl; R³=H; R⁴=OCH₃; R⁵=H; X¹=X²=C₆F₅.

11. Compound selected from one of the following compounds (TBS=t-butyldimethylsilyl):

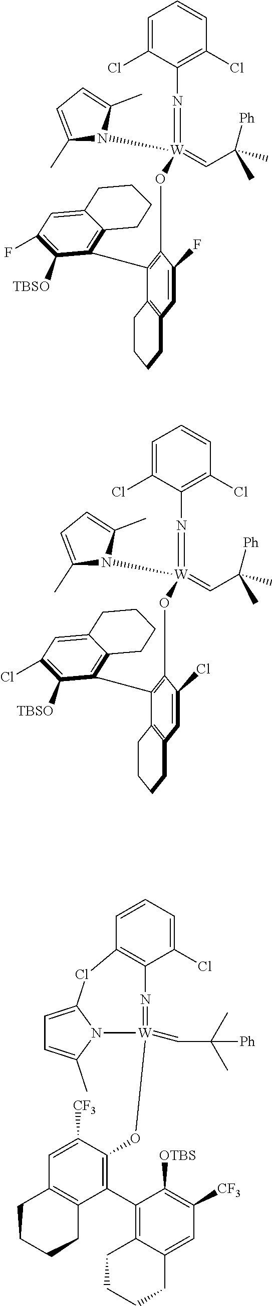

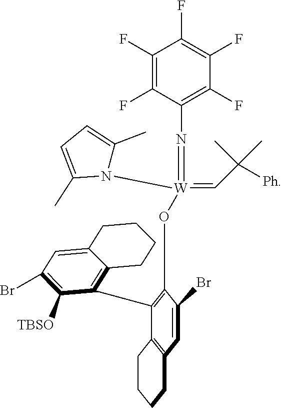

12. Method of performing a metathesis reaction, wherein the metathesis reaction is selected from ethenolysis of an internal olefin, cross-metathesis of an olefin, and a ring-closing metathesis reaction, the method comprising:
performing the metathesis reaction in the presence of a compound of formula (I), (II), (III), (IV) or (VI) as defined in any one of claims 1 to 10 including the disclaimed compound in claim 3.

13. Method of claim 12, wherein ethenolysis is ethenolysis of an unsaturated fatty acid ester, and the cross-metathesis is homo-metathesis of an unsaturated fatty acid ester.

14. Method of performing a metathesis reaction, wherein the metathesis reaction is ethenolysis of an unsaturated fatty acid ester, or the metathesis reaction is homo-metathesis of an unsaturated fatty acid ester, or a ring-dosing reaction, the method comprising:
performing the metathesis reaction in the presence of a compound of formula (V) 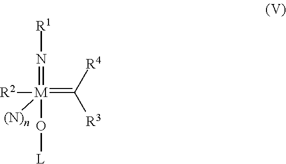
wherein
M=W;
R¹ is phenyl substituted with one or more of halogen or CF₃;
R² is pyrrol-1-yl or indol-1-yl, optionally substituted;
one of R³ and R⁴ is H, and the other is C(CH₃)₂C₆H₅;
LO— is 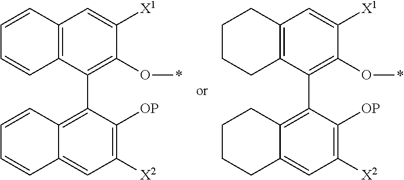
wherein X¹ and X² are independently selected from halogen, CF₃ and C₆F₆; or
X¹=X²=halogen, CF₃ or C₆F₅;
P is C₁-C₆ alkyl, or a silyl group; and
N is a neutral ligand bound to M, wherein n is 0, 1 or 2.

15. Method of any one of claims 13 to 14, wherein said unsaturated fatty acid ester is a natural oil.

16. Method of any one of claims 13 to 15, wherein said unsaturated fatty acid ester is a methyl ester (FAME).

17. Method of claim 16, wherein the methyl ester (FAME) is methyl oleate or methyl linolate or methyl linolenoate or a mixture of two or three thereof.

18. Method of any one of claims 12 to 17, wherein said olefin to be metathesized is purified prior to metathesis by subjecting same to a trialkyl aluminium compound.

19. Compound of formula (I), (II), (III), (IV), or (VI) as defined in any one of claims 1 to 10, wherein LO— is racemic, or
method of claim 12 or 13, wherein in the compound of formula (I), (II), (III), (IV), or (VI) LO— is racemic; or
method of any one of claims 14 to 18, wherein in the compound of formula (V) LO— is racemic.

20. Composition comprising a compound of formula (I), (II), (III), (IV), (V) or (VI) and an olefin to be metathesized, wherein the olefin to be metathesized has been subjected to a trialkyl aluminium compound prior to metathesis.

21. Composition of claim 20, wherein in the compound of formula (I), (II), (III), (IV), (V) or (VI) LO— is racemic.

22. Compound of formula (I), (II), (III), (IV), or (VI) as defined in any one of claims 1 to 10 wherein the neutral ligand N is selected from a nitrile, a phosphine, or a pyridine; or
method of any one of claims 12 to 13, wherein in the compound of formula (I), (II), (III), (IV), or (VI) the neutral ligand N is selected from a nitrile, a phosphine, or a pyridine; or
method of any one of claims 14 to 18, wherein in the compound of formula (V) the neutral ligand N is selected from a nitrile, a phosphine, or a pyridine; or
composition of claim 20 or 21, wherein in the compound of formula (I), (II), (III), (IV), (V) or (VI) the neutral ligand N is selected from a nitrile, a phosphine, or a pyridine.

23. Compound, method or composition of claim 22, wherein the pyridine is 2,2′-dipyridine or 1,10-phenanthroline, and wherein n=1.

24. Compound, method or composition of claim 23, wherein the compound of formula (III) is

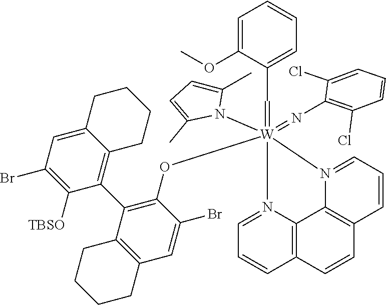

25. Compound 4, wherein the aryloxy-ligand is in racemic form.
